(19) United States
(12) Patent Application Publication
(10) Pub. No.: US 2022/0202803 A1
(43) Pub. Date: Jun. 30, 2022

(54) COMBINATION THERAPIES INCLUDING INHIBITORS OF DIHYDROOROTATE DEHYDROGENASE

(71) Applicant: Clear Creek Bio, Inc., Cambridge, MA (US)

(72) Inventors: Vikram S. Kumar, Boston, MA (US); Andrew D. Levin, Newton, MA (US); David P. Hesson, Malvern, PA (US)

(21) Appl. No.: 17/605,885

(22) PCT Filed: Apr. 21, 2020
(86) PCT No.: PCT/US2020/029080

§ 371 Date: Oct. 22, 2021

Related U.S. Application Data
(60) Provisional application No. 62/838,730, filed on Apr. 25, 2019.
(60) Provisional application No. 62/859,319, filed on Jun. 10, 2019.

(51) Int. Cl. A61K 31/47; A61K 31/42; A61K 31/277; A61K 31/7056; A61K 31/343; A61K 31/635; A61P 35/00

(52) U.S. Cl. CPC A61K 31/47; A61K 31/42; A61K 31/277; A61P 35/00; A61K 31/343; A61K 31/635; A61K 31/7056

(57) ABSTRACT

The invention provides therapeutic methods that include providing to a subject an inhibitor of dihydroorotate dehydrogenase (DHODH) and a second therapeutic agent. The methods are useful for treating cancers, such as leukemias.

### CROSS-REFERENCE TO RELATED APPLICATIONS

This application claims the benefit of, and priority to, U.S. Provisional Patent Application No. 62/838,730, filed Apr. 25, 2019, and U.S. Provisional Patent Application No. 62/859,319, filed Jun. 10, 2019, the contents of each which are incorporated by reference.

### FIELD OF THE INVENTION

The invention generally relates to therapeutic compositions and methods.

### BACKGROUND

Each year about 1.7 million people in the United States are diagnosed with cancer and about 600,000 die from it. Because proliferating cancer cells show substantially different metabolic needs compared to normal differentiated cells, agents that inhibit production of nutrients required specifically or preferentially by cancer cells are candidates for anti-cancer drugs. For example, DNA replication occurs at a much higher rate in cancer cells than in most normal cells, so compounds that limit the ability of cells to replicate DNA are useful for treating cancer. However, it is challenging to identify agents that block proliferation of cancer cells without causing serious harm to healthy cells. Another problem is that many cancer treatments lose their effectiveness over time as cancer cells become resistant to a particular drug. Consequently, existing treatment are inadequate for many types of cancer.

### SUMMARY

The invention provides methods of treating conditions, such as cancer, using combination therapies that include an inhibitor of dihydroorotate dehydrogenase (DHODH) to disrupt pyrimidine synthesis and a second therapeutic agent that targets a different pathway critical for the growth of cancer cells. The invention recognizes that DHODH inhibitors are useful to treat cancer because cancer cells require an abundant supply of pyrimidines to replicate their DNA. The invention further recognizes that the value of DHODH inhibitors in cancer treatments is enhanced by combining such inhibitors with other agents that block growth of cancer cells by a different mechanism. Because cancer cells are highly adaptable, they can develop resistance to a particular agent or even to multiple agents that target the same pathway. The combination therapies of the invention overcome this problem by providing multiple agents that prevent growth of cancer cells by interfering with distinct pathways. The invention includes both multistage therapies in which a DHODH inhibitor is administered to a patient in one stage and a second therapeutic agent is administered in another stage and concurrent therapies in which the two agents are provided simultaneously or in overlapping periods.

The methods of the invention unlock the therapeutic potential of DHODH inhibitors as anti-cancer agents. The therapies include stages during which a DHODH inhibitor is provided to a subject. Such stages include periods of sustained inhibition of DHODH as well as periods in which the DHODH inhibitor is withheld from the subject. Sustained inhibition of DHODH is necessary to achieve killing of cancer cells, while drug-free periods allow sufficient restoration of pyrimidine synthesis to support survival of healthy cells. The therapies also include stages during which a second agent prevents proliferation of cancer cells without affecting pyrimidine synthesis. For example, the second agent may interfere with the mechanics of DNA replication, curb the signal that triggers cells to divide, or selectively kill cancer cells. By switching agents, the therapies decrease the likelihood that cancer cells will develop chemoresistance to an individual agent. Moreover, the combination treatments maintain a high level of efficacy even if cancer cells do develop chemoresistance to one agent.

In an aspect, the invention provides combination therapies for treatment of cancer in a subject. The combination therapies include a dihydroorotate dehydrogenase (DHODH) inhibitor and another agent.

The DHODH inhibitor may be brequinar, leflunomide, teriflunomide, or an analog, derivative, prodrug, micellar formulation, sustained release formulation, or salt of any of the aforementioned compounds.

The other agent may be an inhibitor of inosine monophosphate dehydrogenase (IMPDH), an inhibitor of hypoxanthine-guanine phosphoribosyltransferase (HGPRT), an inhibitor of dihydrofolate reductase (DHFR), an inhibitor of Bcl-2, an agent that targets a cell-surface marker, an inhibitor of a cytokine receptor, an inhibitor of DNA methylation, an inhibitor of a DNA polymerase, a DNA alkylating agent, an analog of a nucleoside or nucleobase, an inhibitor of a topoisomerase, an inhibitor of a Hedgehog signaling pathway, an inhibitor of a tyrosine kinase, an inhibitor of a phosphoinositide 3-kinase (PI3-kinase), an inhibitor of eukaryotic initiation factor 4A (eIF4A), or an asparaginase.

The IMPDH inhibitor may be mizoribine, mycophenolic acid, ribavirin, selenazofurin, taribavirin, tiazofurin, or an analog, derivative, prodrug, micellar formulation, sustained release formulation, or salts of any of the aforementioned compounds.

The HGPRT inhibitor may be 6-mercaptopurine 1,3-dinitroadamantane, acyclovir, or pentamidine.

The DFHR inhibitor may be aminopterin, methotrexate, pemetrexed, pralatrexate, raltitrexed, or trimetrexate.

The Bcl-2 inhibitor may be ABT-737, navitoclax, oblimersen, or venetoclax.

The agent that targets a cell-surface marker may target CD19, CD20, CD22, CD33, or CD52. The CD19-targeting agent may be blinatumomab. The CD20-targeting agent may be obinutuzumab, ofatumumab, or rituximab. The CD22-targeting agent may be inotuzumab ozogamicin, or moxetumomab pasudotox. The CD33-targeting agent may be 161533 TriKE fusion protein, ²²⁵Ac-lintuzumab, AMG 330, AMG 673, AMV564, BI 836858, gemtuzumab ozogamicin, IMGN779, or vadastuximab talirine (SGN-CD33A). The CD52-targeting agent may be alemtuzumab.

The cytokine receptor inhibitor may be anakinra, basiliximab, or daclizumab.

The DNA methylation inhibitor may be azacitidine or azacitidine or decitabine.

The DNA polymerase inhibitor may be cladribine, clofarabine, cytarabine, decitabine, deoxyadenosine, deoxycytidine, fludarabine, gemcitabine, nelarabine, pentostatin, or tezacitibine.

The DNA alkylating agent may be bendamustine, busulfan, chlorambucil, cyclophosphamide, or mechlorethamine.

The analog of a nucleoside or nucleobase may be azacitidine, cladribine, or thioguanine.

The topoisomerase inhibitor may be amsacrine, aurintricarboxylic acid, camptothecin, daunorubicin, doxorubicin. EGCG, ellipticine, etoposide (VP-16), genistein, HU-331, idarubicin, irinotecan, lamellarin D, mitoxantrone, quercetin, resveratrol, teniposide, or topotecan.

The inhibitor of a Hedgehog signaling pathway may be cyclopamine, GANT61, glasdegib, LDE225 (sonidegib/erismodegib), or vismodegib (GDC-0449).

The tyrosine kinase inhibitor may be AC220, bosutinib, dasatinib, gilteritinib, KW-2449, ibrutinib, imatinib, lestaurtinib (CEP-701), midostaurin (PKC412), nilotinib, ponatinib, sorafenib, sunitinib, and tandutinib (MILN518).

The PI3-kinase inhibitor may be duvelisib or idelalisib.

The eIF4A inhibitor may be eFT226, elatol, hippuristanol, pateamine A, rocaglamide, or silvestrol.

The other agent may be an asparaginase, hydroxyurea, omacetaxine, prednisone, or vincristine.

The other agent may be an analog, derivative, prodrug, micellar formulation, sustained release formulation, or salt of any of the aforementioned agents.

The DHODH inhibitor may be brequinar or an analog, derivative, prodrug, micellar formulation, sustained release formulation, or salt thereof, and the second agent may be ribavirin or an analog, derivative, prodrug, micellar formulation, sustained release formulation, or salt thereof. The DHODH inhibitor may be brequinar or an analog, derivative, prodrug, micellar formulation, sustained release formulation, or salt thereof, and the second agent may be venetoclax or an analog, derivative, prodrug, micellar formulation, sustained release formulation, or salt thereof.

In another aspect, the invention provides methods of treating cancer in a subject. The methods include providing to a subject having cancer a dihydroorotate dehydrogenase (DHODH) inhibitor and another agent.

The DHODH inhibitor may be any of the DHODH inhibitors described above.

The other agent may be any of the agents described above.

The DHODH inhibitor and the other agent may be provided according to the same dosing regimen. The DHODH inhibitor and the other agent may be provided according to different dosing regimens.

Each of the DHODH inhibitor and the other agent may independently be provided intravenously, orally, enterally, parenterally, dermally, transdermally, by injection, subcutaneously, pulmonarily, or with or on an implantable medical device.

The cancer may be leukemia, e.g., acute myeloid leukemia (AML) or acute lymphoblastic leukemia (ALL), PTEN null prostate cancer, lung cancer, e.g., small cell lung cancer or non-small cell lung cancer, breast cancer, e.g., triple negative breast cancer (TNBC), glioma, multiple myeloma, neuroblastoma, or adult T cell leukemia/lymphoma (ATLL).

The method may include providing the DHODH inhibitor during a first stage and providing the other agent during a second stage. The first stage and the second stage may overlap partially, may overlap completely, or may not overlap at all. The first stage and the second stage may be concurrent. The first stage and the second stage may be separated by a gap during which neither the DHODH inhibitor nor the other agent is provided to the subject. The first stage and the second stage may be sequential. The first stage and the second stage may make up a cycle, and the method may include multiple cycles. For example, the method may include two, three, four, five, six, or more cycles.

Each stage may independently include a phase in which the DHODH inhibitor or other agent is provided to the subject and a phase during which the DHODH inhibitor or other agent is withheld from the subject. The phase in which the DHOHDH inhibitor or other agent is provided to the subject may be about 24 hours, about 36 hours, about 48 hours, about 60 hours, about 72 hours, about 84 hours, about 96 hours, about 5 days, about 6 days, about 7 days, about 8 days, about 9 days, about 10 days, about 12 days, about 2 weeks, about 3 weeks, about 4 weeks, about 5 weeks, about 6 weeks, about 8 weeks, from about 1 day to about 2 days, from about 1 day to about 3 days, from about 1 day to about 4 days, from about 1 day to about 5 days, from about 1 day to about 6 days, from about 1 day to about 7 days, from about 1 day to about 8 days, from about 1 day to about 9 days, from about 1 day to about 10 days, from about 1 day to about 12 days, from about 1 day to about 14 days, from about 1 day to about 3 weeks, or from about 1 day to about 4 weeks. The phase in which the DHODH inhibitor or other agent is withheld from the subject may be about 24 hours, about 36 hours, about 48 hours, about 60 hours, about 72 hours, about 84 hours, about 96 hours, about 5 days, about 6 days, about 7 days, about 8 days, about 9 days, about 10 days, about 12 days, about 2 weeks, about 3 weeks, about 4 weeks, about 5 weeks, about 6 weeks, about 8 weeks, about 2 months, about 3 months, about 4 months, about 5 months, about 6 months, from about 1 day to about 2 days, from about 1 day to about 3 days, from about 1 day to about 4 days, from about 1 day to about 5 days, from about 1 day to about 6 days, from about 1 day to about 1 week, from about 1 day to about 2 weeks, from about 1 day to about 3 weeks, from about 1 day to about 4 weeks, from about 1 day to about 6 weeks, from about 1 day to about 8 weeks, from about 1 day to about 2 months, from about 1 day to about 3 months, from about 1 day to about 4 months, from about 1 day to about 5 months, or from about 1 day to about 6 months.

Each stage and the gap between stages may independently have a duration of about 24 hours, about 36 hours, about 48 hours, about 60 hours, about 72 hours, about 84 hours, about 96 hours, about 5 days, about 6 days, about 7 days, about 8 days, about 9 days, about 10 days, about 12 days, about 2 weeks, about 3 weeks, about 4 weeks, about 5 weeks, about 6 weeks, about 8 weeks, about 2 months, about 3 months, about 4 months, about 5 months, about 6 months, about 9 months, about 12 months, from about 1 day to about 2 days, from about 1 day to about 3 days, from about 1 day to about 4 days, from about 1 day to about 5 days, from about 1 day to about 6 days, from about 1 day to about 1 week, from about 1 day to about 2 weeks, from about 1 day to about 3 weeks, from about 1 day to about 4 weeks, from about 1 day to about 6 weeks, from about 1 day to about 8 weeks, from about 1 day to about 2 months, from about 1 day to about 3 months, from about 1 day to about 4 months, from about 1 day to about 5 months, or from about 1 day to about 6 months.

The duration of each stage may be determined by a response in the subject. The duration may be determined by the subject's development of resistance to the DHODH inhibitor or other agent. Resistance may be assessed by a change in a level of a biomarker in the subject. For example, the biomarker for resistance to the DHODH inhibitor may be N-carbamoylaspartate, dihydroorotate, orotate, orotidine 5′-monophosphate (OMP), or uridine monophoshpate (UMP).

The biomarker for resistance to an IMPDH inhibitor may be guanosine triphosphate.

The duration may be determined by a change in peripheral blood cell count, bone marrow blast count, tumor burden, or clinical outcome. A change in clinical outcome may include a change from one to another of the following disease states: active disease, minimal residual disease, remission, and complete molecular remission.

The choice of second agent may be determined by a response of the subject, such as any of the responses described above.

### BRIEF DESCRIPTION OF THE DRAWINGS

FIG. 1 is a graph showing mean tumor volume in mice treated either with vehicle, brequinar, ribavirin, and combinations of brequinar and ribavirin.

FIG. 2 is a graph showing the mean tumor volume in mice after treatment with vehicle, brequinar, venetoclax, and combinations of brequinar and venetoclax.

### DETAILED DESCRIPTION

The invention provides methods of treating conditions, such as cancer, using combination therapies. One component of the therapies is an inhibitor of dihydroorotate dehydrogenase (DHODH). DHODH is a critical enzyme in synthesis of pyrimidines, which are required for DNA replication. Because cancer cells divide rapidly, sustained inhibition of DHODH is more toxic to cancer cells than to normal cells. However, a minimum level of pyrimidine synthesis is required by normal cells too. Consequently, administration of DHODH inhibitors, such as brequinar, to achieve therapeutic benefit without causing toxicity to healthy cells is challenging.

The therapeutic methods of the invention solve that problem by employing a second agent that targets a different pathway critical for the growth of cancer cells. The methods include multistage therapies in which a DHODH inhibitor is administered to a patient in one stage and a second therapeutic agent is administered in another stage. By switching between therapeutic agents, prolonged exposure to a DHODH inhibitor is avoided. Thus, the methods overcome the problems associated with long-term inhibition of DHODH, such as toxicity and chemoresistance.

### DHODH Inhibitors and Markers of DHODH Inhibition

DHODH is a key enzyme in the pyrimidine biosynthesis pathway. Pyrimidine biosynthesis involves a sequence of step enzymatic reactions that result in the conversion of glutamine to uridine monophosphate as shown below:

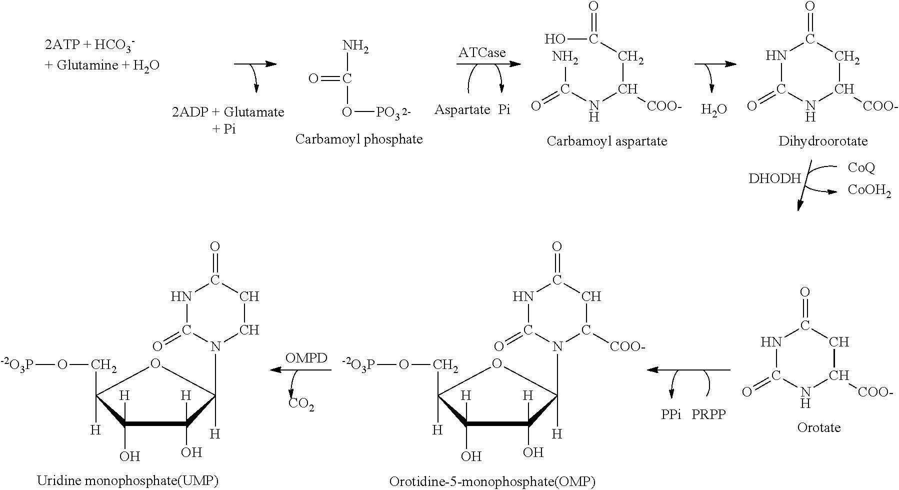

Several of the enzymes in the pyridine synthesis pathway are targets of drugs or drug candidates. For example, aspartate carbamoyltransferase (also known as aspartate transcarbamoylase or ATCase), which catalyzes the conversion of carbamoyl phosphate to carbamoyl aspartate, is inhibited by PALA (N-phosphoacetyl-L-aspartate); dihydroorotate dehydrogenase (DHODH), which catalyzes conversion of dihydroorotate (DHO) to orotate, is inhibited by brequinar, leflunomide, and teriflunomide; and orotidine monophosphate decarboxylase (OMPD), which catalyzes conversion of orotidine monophosphate (OMP) to uridine monophosphate (UMP), is inhibited by pyrazofurin.

Exemplary DHODH inhibitors useful for embodiments of the invention are provided below.

Brequinar, 6-fluoro-2-(2′-fluoro-1,1′ biphenyl-4-yl)-3-methyl-4-quinoline carboxylic acid, has the following structure:

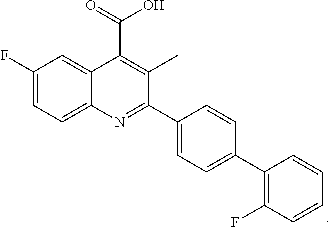 [SMILES: Cc1c(-c2ccc(-c3ccccc3F)cc2)nc2ccc(F)cc2c1C(=O)O]

Brequinar and related compounds are described in, for example, U.S. Pat. Nos. 4,680,299 and 5,523,408, the contents of which are incorporated herein by reference. The use of brequinar to treat leukemia is described in, for example, U.S. Pat. No. 5,032,597 and International Publication No. WO 2017/037022, the contents of which are incorporated herein by reference.

Leflunomide, N-(4′-trifluoromethylphenyl)-5-methylisoxazole-4-carboxamide (I), is described in, for example, U.S. Pat. No. 4,284,786, the contents of which are incorporated herein by reference.

Teriflunomide, 2-cyano-3-hydroxy-N-[4-(trifluoromethyl)phenyl]-2-butenamide, is described in, for example, U.S. Pat. No. 5,679,709, the contents of which are incorporated herein by reference.

The DHODH inhibitor, such as any of the aforementioned compounds, may be provided as an analog, derivative, prodrug, micellar formulation, sustained release formulation, or salt of the pharmacologically active compound.

Because many of the enzymes involved in pyrimidine or purine biosynthesis are the targets of known inhibitors, metabolites in these pathways can serve as indicators of engagement of therapeutic agents with their targets. For example, the utility of DHO as an indicator of target engagement by DHODH inhibitors has been described in co-owned, co-pending U.S. Application No. 62/682,419, the contents of which are incorporated herein by reference. One advantage of DHO is that cell membranes are permeable to the molecule. DHODH is localized to the mitochondrial inner membrane within cells, making direct measurement of enzyme activity difficult. However, DHO, which accumulates when DHODH is inhibited, diffuses out of cells and into the blood, which can be easily sampled. DHO is also sufficiently stable that levels of the metabolite can be measured reliably. Thus, by analyzing levels of DHO in blood or blood products, one can readily assess target engagement of a DHODH inhibitor.

### Other Therapeutic Agents

The second therapeutic agent, i.e., the therapeutic agent other than a DHODH inhibitor, may be any agent that targets a second pathway, i.e., a pathway other than pyrimidine synthesis, that is critical for the growth of cancer cells. Some exemplary agents and pathways are provided below. It will be understood that these agents and pathways are provided only to illustrate embodiments of the invention and that the scope of the invention is not limited by the examples provided.

Agents that inhibit purine synthesis are useful in methods of the invention. As indicated above, cancer cells have high rates of DNA replication, and DNA replication requires an ample supply of purines as well as pyrimidines. Consequently, several enzymes involved in synthesis of purines, such as guanosine, are also of therapeutic interest. Some of the key reactions involved in synthesis of guanine nucleotides are shown below:

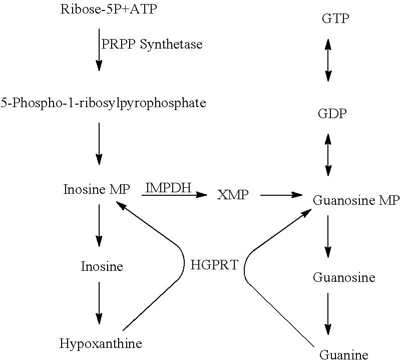

The multi-step conversion of ribose-5-phosphate (ribose-5P) to inosine monophosphate shown in the upper left portion of the diagram is required for all de novo purine synthesis. For synthesis of guanine nucleotides, such guanosine triphosphate (GTP) and deoxyguanosine triphosphate (dGTP), inosine monophosphate dehydrogenase (IMPDH) catalyzes the nicotinamide adenine dinucleotide (NADY)-dependent oxidation of inosine monophosphate (IMP) to xanthosine monophosphate (XMP). Guanosine monophosphate synthetase (GMPS) then converts XMP to guanosine monophosphate. For synthesis of GTP, shown in the upper right portion of the diagram, two more phosphate moieties are added stepwise to create guanosine diphosphate (GDP) first and then GTP. For synthesis of dGTP, a building block for DNA replication, GDP is converted to deoxyguanosine diphosphate (dGDP), which is then phosphorylated to produce dGTP.

Purines can also be synthesized from the components of degraded macromolecules via salvage pathways. The lower half of the diagram shows that IMP and GMP can be broken down to hypoxanthine and guanine, respectively, via multiple steps. However, hypoxanthine-guanine phosphoribosyltransferase (HGPRT) reverses both series of reactions to regenerate IMP and GMP under appropriate conditions. Thus, HGPRT bypasses IMPDH to synthesize guanine nucleotides via the guanine salvage pathway. Certain tissues and organs are unable to undergo de novo synthesis of purines and rely exclusively on salvage pathways to supply needed nucleotides.

Hypoxanthine can be sequentially converted by xanthine oxidase (XO) first to xanthine and then to uric acid. Inhibition of IMPDH can therefore lead to a high concentration of uric acid in the blood, which may cause medical problems, including kidney stones, gout, and diabetes. Consequently, the therapeutic use of IMPDH inhibitors is usually accompanied by administration of an inhibitor of XO to prevent accumulation of uric acid in the body.

Humans have two IMPDH isozymes that have similar enzymatic characteristics. The differential roles of the two isozymes are not well understood.

Given the role of IMPDH in synthesis of guanine nucleotides, metabolites in guanine nucleotide synthesis pathways are useful indicators for engagement of IMPDH inhibitors with their target. For example, inhibition of IMPDH prevents de novo synthesis of GTP, so decreased GTP levels are indicative of the degree of IMPDH inhibition in cells capable of de novo GTP synthesis. In addition, IMPDH inhibition results in the accumulation of the substrate IMP, which is then converted to hypoxanthine. As indicated above, conversion of hypoxanthine to uric acid can be blocked by providing inhibitors of XO. Therefore, in subjects that have received inhibitors of both IMPDH and XO, increased levels hypoxanthine are indicative of the degree of IMPDH inhibition.

The second therapeutic agent in methods of the invention may be an IMPDH inhibitor. IMPDH inhibitors are known in the art and described in, for example, Cuny, G. D., et al., Inosine-5′-monophosphate dehydrogenase (IMPDH) inhibitors: a patent and scientific literature review (2002-2016), Expert Opin Ther Pat., 2017, June; 27(6):677-690. doi: 10.1080/13543776.2017.1280463, the contents of which are incorporated herein by reference.

In some embodiments, the IMPDH inhibitor is tiazofurin. Tiazofurin, which has the systematic name 2-β-D-ribofuranosylthiazole-4-carboxamide, has the following structure:

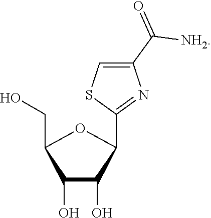

Tiazofurin and methods for making it are known in the art and described in, for example, U.S. Pat. Nos. 4,451,648 and 6,613,896, the contents of which are incorporated herein by reference. Tiazofurin analogs, their activity against tumors, and methods of making them are described in, for example, Popsavin, et al., Synthesis and antiproliferative activity of two new tiazofurin analogues with 2′-amido functionalities, Bioorg. Med. Chem. Lett. 16 (2006) 2773-2776, doi: 10.1016/j.bmcl.2006.02.001; and Popsavin, et al., Synthesis and in vitro antitumor activity of tiazofurin analogues with nitrogen functionalities at the C-2′ position, European Journal of Medicinal Chemistry 111 (2016) 114e125. doi: 10.1016/j.ejmech.2016.01.037, the contents of each of which are incorporated herein by reference.

Tiazofurin is converted inside cells to thiazole-4-carboxamide adenine dinucleotide (TAD). TAD, an analog of NAD⁺, is the form of the compound that interacts with IMDH to block its activity.

Another IMPDH inhibitor is the non-reversible inhibitor mycophenolic acid. Mycophenolic acid has the following structure:

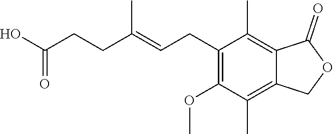 [SMILES: COc1c(C)c2c(c(C)c1C/C=C(\C)CCC(=O)O)C(=O)OC2]

Uses, side effects, and the mechanism of action of mycophenolate are known in the art and described in, for example, Kitchin, J. E., et al., (1997) “Rediscovering mycophenolic acid: A review of its mechanism, side effects, and potential uses” Journal of the American Academy of Dermatology. 37 (3): 445-449. doi:10.1016/S0190-9622(97)70147-6; and Pharmacology North American Edition. Lippincott Williams & Wilkins. 2014, p. 625, ISBN 978-1-4511-9177-6, the contents of each of which are incorporated herein by reference. Salts, derivatives, analogs, and prodrugs of mycophenolic acid are known in the art and described in, for example, Mele T. S., and Halloran, P. F., The use of mycophenolate mofetil in transplant recipients. Immunopharmacology. 2000; 47:215-245; International Publication No. WO 2004/096287A2; and U.S. Pat. No. 7,427,636, the contents of each of which are incorporated herein by reference.

Many other inhibitors of IMPDH are known in the art. For example and without limitation, other IMPDH inhibitors include AS2643361, EICAR, FF-10501, mizoribine, ribavirin, selenazofurin, SM-108, taribavirin, VX-148, VX-497, and VX-944. For example and without limitation, other IMPDH inhibitors include ribavirin, mizoribine, selenazofurin, and taribavirin. Other IMPDH inhibitors are described in Gebeyehu, G., et al., Ribavirin, Tiazofurin, and Selenazofurin: Mononucleotides and Nicotinamide Adenine Dinucleotide Analogues. Synthesis, Structure, and Interactions with IMP Dehydrogenase, J. Med. Chem. 1985, 28, 99-105; and U.S. Pat. Nos. 5,807,876; 6,344,465; 6,395,763; 6,399,773; 6,420,403; 6,518,291; 6,541,496; 6,617,323; 6,624,184; 6,653,309; 6,825,224; 6,867,299; 6,919,335; 6,967,214; 7,053,111; 7,060,720; 7,087,642; 7,205,324; 7,329,681; 7,432,290; 7,777,069; and 7,989,498, the contents of each of which are incorporated herein by reference. Some IMPDH inhibitors have additional modes of action, such as ribavirin which is also a proposed inhibitor of eukaryotic translation initiation factor 4E (eIF4E).

The second therapeutic agent may be an inhibitor of HGPRT. HGPRT inhibitors include 6-mercaptopurine 1,3-dinitroadamantane, acyclovir, and pentamidine.

The second therapeutic agent may be an inhibitor of dihydrofolate reductase (DHFR), which reduces dihydrofolic acid to tetrahydrofolic acid in the purine salvage pathway. DHFR inhibitors include aminopterin, methotrexate, pemetrexed, pralatrexate, raltitrexed, and trimetrexate.

The second therapeutic agent may be an inhibitor of Bcl-2. Bcl-2 inhibitors include ABT-737, navitoclax, oblimersen, and venetoclax.

The second therapeutic agent may be an agent that targets a cell surface marker. For example, the agent may target a cluster of differentiation (CD) marker, such as CD19, CD20, CD22, CD33, or CD52. CD19-targeting agents include blinatumomab. CD20-targeting agents include obinutuzumab, ofatumumab, and rituximab. CD22-targeting agents include inotuzumab ozogamicin, and moxetumomab pasudotox. CD33-targeting agents include 161533 TriKE fusion protein, ²²⁵Ac-lintuzumab, AMG 330, AMG 673, AMV564, BI 836858, gemtuzumab ozogamicin, IMGN779, and vadastuximab talirine (SGN-CD33A). CD52-targeting agents include alemtuzumab.

The second therapeutic agent may be an inhibitor of a cytokine receptor. Cytokine receptor inhibitors include anakinra, basiliximab, and daclizumab.

The second therapeutic agent may be an agent that interferes with DNA replication. The agent may interfere with DNA replication by inhibiting DNA methylation, inhibiting DNA polymerase, alkylating bases in DNA, direct incorporation into DNA, inhibiting topoisomerase, or any combination of such mechanisms. DNA methylation inhibitors include azacitidine and decitabine. DNA polymerase inhibitors include cladribine, clofarabine, cytarabine, decitabine, deoxyadenosine, deoxycytidine, fludarabine, gemcitabine, nelarabine, pentostatin, and tezacitibine. DNA alkylating agents include bendamustine, busulfan, chlorambucil, cyclophosphamide, and mechlorethamine. Nucleoside analogs or nucleobase analogs that become incorporated into DNA include azacitidine, cladribine, and thioguanine. Topoisomerase inhibitors include amsacrine, aurintricarboxylic acid, camptothecin, daunorubicin, doxorubicin. EGCG, ellipticine, etoposide (VP-16), genistein, HU-331, idarubicin, irinotecan, lamellarin D, mitoxantrone, quercetin, resveratrol, teniposide, and topotecan.

The second therapeutic agent may be an inhibitor of a Hedgehog signaling pathway. Inhibitors of Hedgehog signaling pathways include cyclopamine, GANT61, glasdegib, LDE225 (sonidegib/erismodegib), and vismodegib (GDC-0449).

The second therapeutic agent may be a tyrosine kinase inhibitor. Tyrosine kinase inhibitors include AC220, bosutinib, dasatinib, gilteritinib, KW-2449, ibrutinib, imatinib, lestaurtinib (CEP-701), midostaurin (PKC412), nilotinib, ponatinib, sorafenib, sunitinib, and tandutinib (MLN518).

The second therapeutic agent may be a PI3 kinase inhibitor, such as duvelisib or idelalisib.

The second therapeutic agent may be an eIF4A inhibitor, such as eFT226, elatol, hippuristanol, pateamine A, rocaglamide, or silvestrol.

The second therapeutic agent may be an asparaginase.

The second therapeutic agent may be hydroxyurea, omacetaxine, prednisone, or vincristine.

The second therapeutic agent, such as any of the aforementioned agents, may be provided as an analog, derivative, prodrug, micellar formulation, sustained release formulation, or salt of the pharmacologically active compound.

### Determining a Dosing Regimen for the DHODH Inhibitor

Methods of the invention may include determining a dosing regimen of a DHODH inhibitor for a subject. The dosing regimen may include a dose, i.e., an amount, of the DHODH inhibitor that should be administered. The dosing regimen may include a time point for administration of a dose of the DHODH inhibitor to the subject. The dosing regimen may be based on one or more measured levels of DHO in a sample obtained from the subject, which allows the dosing regimen to be tailored to an individual subject, e.g., a patient. Consequently, the methods of the invention provide customized dosing regimens that account for variability in pharmacokinetic properties, i.e., metabolism of the active pharmaceutical ingredient (API) by the subject, and pharmacodynamics properties, effect of the API on its target, among individuals.

The dosing regimen may be determined by comparing a measured level of DHO in a sample obtained from a subject to a reference that provides an association between the measured level and a recommended dosage adjustment of the DHODH inhibitor. For example, the reference may provide a relationship between administration of the DHODH inhibitor and levels of DHO in the subject. The relationship can be empirically determined from a known dose and time of administration of the DHODH inhibitor and measured levels of DHO at one or more subsequent time points. The reference may include a relationship between measured levels of the DHODH inhibitor or a metabolic product of the DHODH inhibitor and measured levels of DHO.

From the comparison between the measured level of DHO and the reference, a dosing regimen may then be determined. The dosing regimen may include a dosage of the DHODH inhibitor, a time for administration of the dosage, or both. The dosing regimen may be determined de novo, or it may comprise an adjustment to a previous dosing regimen, such as an adjustment in the dosage, the interval between administration of dosages, or both.

The dosing regimen is designed to deliver the DHODH inhibitor to the subject in an amount that achieves a therapeutic effect. The therapeutic effect may be a sign or symptom of a disease, disorder, or condition. The therapeutic effect may be inhibition of an enzyme in the metabolic pathway, or it may be a change in an indicator of inhibition of an enzyme in a metabolic pathway. The indicator may be DHO in the pathway, and the therapeutic effect may be an increase or decrease in levels of DHO. The therapeutic effect may be a decrease in number of cancer cells, a decrease in proliferation of cancer cells, an increase in differentiation of pre-cancerous cells, such as myeloblasts, complete remission of cancer, complete remission with incomplete hematologic recovery, morphologic leukemia-free stat, or partial remission. Increased differentiation of myeloblasts may be assessed by one or more of expression of CD14, expression of CD11b, nuclear morphology, and cytoplasmic granules.

The dosing regimen may ensure that levels of DHO are raised or maintained above a minimum threshold required to achieve a certain effect. For example, the dosing regimen may raise or maintain levels of DHO above a threshold level in the subject for a certain time period. The time period may include a minimum, a maximum, or both. For example, the dosing regimen may raise or maintain levels of DHO above the threshold level for at least 6 hours, 12, hours, 24 hours, at least 48 hours, at least 72 hours, at least 84 hours, at least 96 hours, at least 5 days, at least 6 days, at least 7 days, at least 10 days, at least 2 weeks, or more. The dosing regimen may raise or maintain levels of DHO above the threshold level for not more than 24 hours, not more than 36 hours, not more than 48 hours, not more than 60 hours, not more than 72 hours, not more than 84 hours, not more than 96 hours, not more than 5 days, not more than 6 days, not more than 7 days, not more than 10 days, or not more than 2 weeks. The dosing regimen may raise or maintain levels of DHO above the threshold level for at least 72 hours but not more than 96 hours, for at least 72 hours but not more than 5 days, for at least 72 hours but not more than 6 days, for at least 72 hours but not more than 7 days, for at least 96 hours but not more than 7 days.

The dosing regimen may ensure that levels of DHO do not exceed or are maintained below a maximum threshold that is associated with toxicity. Levels of DHO above a maximum threshold may indicate that the DHODH inhibitor is causing or is likely to cause an adverse event in the subject. For example and without limitation, adverse events include abdominal pain, anemia, anorexia, blood disorder, constipation, diarrhea, dyspepsia, fatigue, fever, granulocytopenia, headache, infection, leukopenia, mucositis, nausea, pain at the injection site, phlebitis, photosensitivity, rash, somnolence, stomatitis, thrombocytopenia, and vomiting.

The dosing regimen may include a time point for administration of one or more subsequent doses to raise or maintain levels of DHO above a minimum threshold level for a certain time period. The time point for administration of a subsequent dose may be relative to an earlier time point. For example, the time point for administration of a subsequent dose may be relative to a time point when a previous dose was administered or a time point when a sample was obtained from a subject.

The dosing regimen may include a schedule for administration of doses. For example, doses may be administered at regular intervals, such as every 24 hours, every 36 hours, every 48 hours, every 60 hours, every 72 hours, every 84 hours, every 96 hours, every 5 days, every 6 days, every week, every 2 weeks, every 3 weeks, or every 4 weeks. Alternatively, doses may be administered according to a schedule that does not require precisely regular intervals. For example, doses may be administered once per week, twice per week, three times per week, four times per week, once per month, twice per month, three times per month, four times per month, five times per month, or six times per month.

For example and without limitation, a dosing regimen for administration of a DHODH inhibitor, such brequinar, e.g., brequinar sodium, to a human subject may be as follows: 100 mg/m², administered intravenously twice weekly; 125 mg/m², administered intravenously twice weekly; 150 mg/m², administered intravenously twice weekly; 200 mg/m², administered intravenously twice weekly; 250 mg/m², administered intravenously twice weekly; 275 mg/m², administered intravenously twice weekly; 300 mg/m², administered intravenously twice weekly; 350 mg/m², administered intravenously twice weekly; 400 mg/m², administered intravenously twice weekly; 425 mg/m², administered intravenously twice weekly; 450 mg/m², administered intravenously twice weekly; 500 mg/m², administered intravenously twice weekly; 550 mg/m², administered intravenously twice weekly; 600 mg/m², administered intravenously twice weekly; 650 mg/m², administered intravenously twice weekly; 700 mg/m², administered intravenously twice weekly; 750 mg/m², administered intravenously twice weekly; 800 mg/m², administered intravenously twice weekly; 100 mg/m², administered intravenously every 72 hours; 125 mg/m², administered intravenously every 72 hours; 150 mg/m², administered intravenously every 72 hours; 200 mg/m², administered intravenously every 72 hours; 250 mg/m², administered intravenously every 72 hours; 275 mg/m², administered intravenously every 72 hours; 300 mg/m², administered intravenously every 72 hours; 350 mg/m², administered intravenously every 72 hours; 400 mg/m², administered intravenously every 72 hours; 425 mg/m², administered intravenously every 72 hours; 450 mg/m², administered intravenously every 72 hours; 500 mg/m², administered intravenously every 72 hours; 550 mg/m², administered intravenously every 72 hours; 600 mg/m², administered intravenously every 72 hours; 650 mg/m², administered intravenously every 72 hours; 700 mg/m², administered intravenously every 72 hours; 750 mg/m², administered intravenously every 72 hours; 800 mg/m², administered intravenously every 72 hours; 100 mg/m², administered intravenously every 84 hours; 125 mg/m², administered intravenously every 84 hours; 150 mg/m², administered intravenously every 84 hours; 200 mg/m², administered intravenously every 84 hours; 250 mg/m², administered intravenously every 84 hours; 275 mg/m², administered intravenously every 84 hours; 300 mg/m², administered intravenously every 84 hours; 350 mg/m², administered intravenously every 84 hours; 400 mg/m², administered intravenously every 84 hours; 425 mg/m², administered intravenously every 84 hours; 450 mg/m², administered intravenously every 84 hours; 500 mg/m², administered intravenously every 84 hours; 550 mg/m², administered intravenously every 84 hours; 600 mg/m², administered intravenously every 84 hours; 650 mg/m², administered intravenously every 84 hours; 700 mg/m², administered intravenously every 84 hours; 750 mg/m², administered intravenously every 84 hours; 800 mg/m², administered intravenously every 84 hours; 100 mg/m², administered intravenously every 96 hours; 125 mg/m², administered intravenously every 96 hours; 150 mg/m², administered intravenously every 96 hours; 200 mg/m², administered intravenously every 96 hours; 250 mg/m², administered intravenously every 96 hours; 275 mg/m², administered intravenously every 96 hours; 300 mg/m², administered intravenously every 96 hours; 350 mg/m², administered intravenously every 96 hours; 400 mg/m², administered intravenously every 96 hours; 425 mg/m², administered intravenously every 96 hours; 450 mg/m², administered intravenously every 96 hours; 500 mg/m², administered intravenously every 96 hours; 550 mg/m², administered intravenously every 96 hours; 600 mg/m², administered intravenously every 96 hours; 650 mg/m², administered intravenously every 96 hours; 700 mg/m², administered intravenously every 96 hours; 750 mg/m², administered intravenously every 96 hours; 800 mg/m², administered intravenously every 96 hours; 100 mg/m², administered orally twice weekly; 125 mg/m², administered orally twice weekly; 150 mg/m², administered orally twice weekly; 200 mg/m², administered orally twice weekly; 250 mg/m², administered orally twice weekly; 275 mg/m², administered orally twice weekly; 300 mg/m², administered orally twice weekly; 350 mg/m², administered orally twice weekly; 400 mg/m², administered orally twice weekly; 425 mg/m², administered orally twice weekly; 450 mg/m², administered orally twice weekly; 500 mg/m², administered orally twice weekly; 550 mg/m², administered orally twice weekly; 600 mg/m², administered orally twice weekly; 650 mg/m², administered orally twice weekly; 700 mg/m², administered orally twice weekly; 750 mg/m², administered orally twice weekly; 800 mg/m², administered orally twice weekly; 100 mg/m², administered orally every 72 hours; 125 mg/m², administered orally every 72 hours; 150 mg/m², administered orally every 72 hours; 200 mg/m², administered orally every 72 hours; 250 mg/m², administered orally every 72 hours; 275 mg/m², administered orally every 72 hours; 300 mg/m², administered orally every 72 hours; 350 mg/m², administered orally every 72 hours; 400 mg/m², administered orally every 72 hours; 425 mg/m², administered orally every 72 hours; 450 mg/m², administered orally every 72 hours; 500 mg/m², administered orally every 72 hours; 550 mg/m², administered orally every 72 hours; 600 mg/m², administered orally every 72 hours; 650 mg/m², administered orally every 72 hours; 700 mg/m², administered orally every 72 hours; 750 mg/m², administered orally every 72 hours; 800 mg/m², administered orally every 72 hours; 100 mg/m², administered orally every 84 hours; 125 mg/m², administered orally every 84 hours; 150 mg/m², administered orally every 84 hours; 200 mg/m², administered orally every 84 hours; 250 mg/m², administered orally every 84 hours; 275 mg/m², administered orally every 84 hours; 300 mg/m², administered orally every 84 hours; 350 mg/m², administered orally every 84 hours; 400 mg/m², administered orally every 84 hours; 425 mg/m², administered orally every 84 hours; 450 mg/m², administered orally every 84 hours; 500 mg/m², administered orally every 84 hours; 550 mg/m², administered orally every 84 hours; 600 mg/m², administered orally every 84 hours; 650 mg/m², administered orally every 84 hours; 700 mg/m², administered orally every 84 hours; 750 mg/m², administered orally every 84 hours; 800 mg/m², administered orally every 84 hours; 100 mg/m², administered orally every 96 hours; 125 mg/m², administered orally every 96 hours; 150 mg/m², administered orally every 96 hours; 200 mg/m², administered orally every 96 hours; 250 mg/m², administered orally every 96 hours; 275 mg/m², administered orally every 96 hours; 300 mg/m², administered orally every 96 hours; 350 mg/m², administered orally every 96 hours; 400 mg/m², administered orally every 96 hours; 425 mg/m², administered orally every 96 hours; 450 mg/m², administered orally every 96 hours; 500 mg/m², administered orally every 96 hours; 550 mg/m², administered orally every 96 hours; 600 mg/m², administered orally every 96 hours; 650 mg/m², administered orally every 96 hours; 700 mg/m², administered orally every 96 hours; 750 mg/m², administered orally every 96 hours; or 800 mg/m², administered orally every 96 hours.

Minimum and maximum threshold levels of a metabolite depend on a variety of factors, such as the type of subject, metabolite, therapeutic agent, and type of sample. Minimum and maximum threshold levels may be expressed in absolute terms, e.g., in units of concentration, or in relative terms, e.g., in ratios relative to a baseline or reference value. For example, the minimum threshold (below which a patient may receive a dose increase or additional dose) could also be calculated in terms of increase from a pre-treatment DHO level or baseline level.

Minimum threshold levels of DHO or orotate in a human plasma sample may be about 0 ng/ml, about 10 ng/mL, about 20 ng/mL, about 50 ng/mL, about 100 ng/mL, about 150 ng/mL, about 200 ng/mL, about 250 ng/mL, about 300 ng/mL, about 350 ng/mL, about 400 ng/mL, about 450 ng/mL, about 500 ng/mL, about 550 ng/mL, about 600 ng/mL, about 650 ng/mL, about 700 ng/mL, about 750 ng/mL, about 800 ng/mL, about 850 ng/mL, about 900 ng/mL, about 950 ng/mL, about 1000 ng/mL, about 1250 ng/ml, about 1500 ng/ml, about 1750 ng/ml, about 2000 ng/ml, about 2500 ng/ml, about 3000 ng/ml, about 3500 ng/ml, about 4000 ng/ml, about 4500 ng/ml, about 5000 ng/ml, about 6000 ng/ml, about 8000 ng/ml, about 10,000 ng/ml, about 12,000 ng/ml, about 15,000 ng/ml, about 20,000 ng/ml, about 25,000 ng/ml, about 30,000 ng/ml, about 40,000 ng/ml, about 50,000 ng/ml, about 75,000 ng/ml, about 100,000 ng/ml, about 150,000 ng/ml, about 200,000 ng/ml, about 300,000 ng/ml, or about 400,000 ng/ml. The minimum threshold may include any value that falls between the values recited above. Thus, the minimum threshold may include any value between 0 ng/ml and 400.00 ng/ml.

Maximum threshold levels of DHO or orotate in a human plasma sample may be about 50 ng/mL, about 100 ng/mL, about 150 ng/mL, about 200 ng/mL, about 250 ng/mL, about 300 ng/mL, about 350 ng/mL, about 400 ng/mL, about 450 ng/mL, about 500 ng/mL, about 550 ng/mL, about 600 ng/mL, about 650 ng/mL, about 700 ng/mL, about 750 ng/mL, about 800 ng/mL, about 850 ng/mL, about 900 ng/mL, about 950 ng/mL, about 1000 ng/mL, about 1250 ng/ml, about 1500 ng/ml, about 1750 ng/ml, about 2000 ng/ml, about 2500 ng/ml, about 3000 ng/ml, about 3500 ng/ml, about 4000 ng/ml, about 4500 ng/ml, about 5000 ng/ml, about 6000 ng/ml, about 8000 ng/ml, about 10,000 ng/ml, about 12,000 ng/ml, about 15,000 ng/ml, about 20,000 ng/ml, about 25,000 ng/ml, about 30,000 ng/ml, about 40,000 ng/ml, about 50,000 ng/ml, about 75,000 ng/ml, about 100,000 ng/ml, about 150,000 ng/ml, about 200,000 ng/ml, about 300,000 ng/ml, about 400,000 ng/ml, or about 500,000 ng/ml. The maximum threshold may include any value that falls between the values recited above. Thus, the maximum threshold may include any value between 50 ng/ml and 500,000 ng/ml.

The minimum or maximum threshold may also be expressed in terms of increase from a pre-treatment DHO level or baseline level.

For example, the minimum threshold of DHO or orotate may be about 1.5 times the baseline level, about 2 times the baseline level, about 2.5 times the baseline level, about 3 times the baseline level, about 4 times the baseline level, about 5 times the baseline level, about 10 times the baseline level, about 20 times the baseline level, about 50 times the baseline level, about 100 times the baseline level, about 200 times the baseline level, about 500 times the baseline level, about 1000 times the baseline level, about 2000 times the baseline level, or about 5000 times the baseline level. The minimum threshold may include any ratio that falls between those recited above. Thus, the minimum threshold may be any ratio between 1.5 times the baseline level and 5000 times the baseline level.

The maximum threshold of DHO or orotate may be about 2 times the baseline level, about 2.5 times the baseline level, about 3 times the baseline level, about 4 times the baseline level, about 5 times the baseline level, about 10 times the baseline level, about 20 times the baseline level, about 50 times the baseline level, about 100 times the baseline level, about 200 times the baseline level, about 500 times the baseline level, about 1000 times the baseline level, about 2000 times the baseline level, about 5000 times the baseline level, or about 10,000 times the baseline level. The maximum threshold may include any ratio that falls between those recited above. Thus, the maximum threshold may be any ratio between 2 times the baseline level and 10,000 times the baseline level.

The DHODH inhibitor may be any DHODH inhibitor, such as any of those described above.

Dosing of the DHODH inhibitor may account for the formulation of the DHODH inhibitor. For example, DHODH inhibitors, such as brequinar, leflunomide, and teriflunomide, may be provided as prodrugs, analogs, derivatives, or salts. Any of the aforementioned chemical forms may be provided in a pharmaceutically acceptable formulation, such as a micellar formulation or sustained release formulation.

Dosage of the DHODH inhibitor also depends on factors such as the type of subject and route of administration. The dosage may fall within a range for a given type of subject and route of administration, or the dosage may adjusted by a specified amount for a given type of subject and route of administration. For example, dosage of brequinar for oral or intravenous administration to a subject, such as human or mouse, may be about 1 mg/kg, about 2 mg/kg, about 5 mg/kg, about 7.5 mg/kg, about 10 mg/kg, about 15 mg/kg, about 20 mg/kg, about 25 mg/kg, about 50 mg/kg, about 75 mg/kg, or about 100 mg/kg. Dosage of brequinar for oral or intravenous administration to a subject, such as human or mouse, may be adjusted by about 1 mg/kg, about 2 mg/kg, about 5 mg/kg, about 7.5 mg/kg, about 10 mg/kg, about 15 mg/kg, about 20 mg/kg, about 25 mg/kg, or about 50 mg/kg. Dosage of brequinar for oral or intravenous administration to an animal subject, such as a human or mouse, may be about 50 mg/m², about 100 mg/m², about 200 mg/m², about 300 mg/m², about 350 mg/m², about 400 mg/m², about 500 mg/m², about 600 mg/m², about 700 mg/m², about 800 mg/m², or about 1000 mg/m². Dosage of brequinar for oral or intravenous administration to an animal subject, such as a human or mouse, may be adjusted by about 50 mg/m², about 100 mg/m², about 200 mg/m², about 300 mg/m², about 350 mg/m², or about 400 mg/m².

### Determining a Dosing Regimen for the Second Therapeutic Agent

Methods of the invention may include determining a dosing regimen for the second therapeutic agent. In certain embodiments in which the second agent is an inhibitor of an enzyme in a metabolic pathway, the dosing regimen is determined based on measured levels of a metabolite in the pathway in an analogous manner to that described above for the DHODH inhibitor. For example, the second therapeutic agent may be an IMPDH inhibitor, and the dosing regimen may be determined based on measured levels of guanosine triphosphate (GTP).

### Multistage Treatments

Methods of the invention may include multistage treatments in which the DHODH inhibitor is provided to the subject during one stage and the second therapeutic agent is provided to the subject during the second stage. For example, the dosing regimen for the DHODH inhibitor may make up the first stage of treatment, and the dosing regimen for the second therapeutic agent may make up the second stage of treatment. The first stage and the second stage may make up a cycle, and the method may include multiple cycles. For example, the method may include two, three, four, five, six, or more cycles.

Because a dosing regimen may include periods when the DHODH inhibitor or second therapeutic agent is not provided to the subject, each stage may be subdivided into “on” phases in which the subject received the DHODH inhibitor or second therapeutic agent and “off” phases in which the DHODH inhibitor or second therapeutic agent is withheld from the subject.

For example, each stage may independently include a phase in which the DHODH inhibitor or other agent is provided to the subject and a phase during which the DHODH inhibitor or other agent is withheld from the subject. The phase in which the DHOHDH inhibitor or other agent is provided to the subject may be about 24 hours, about 36 hours, about 48 hours, about 60 hours, about 72 hours, about 84 hours, about 96 hours, about 5 days, about 6 days, about 7 days, about 8 days, about 9 days, about 10 days, about 12 days, about 2 weeks, about 3 weeks, about 4 weeks, about 5 weeks, about 6 weeks, about 8 weeks, from about 1 day to about 2 days, from about 1 day to about 3 days, from about 1 day to about 4 days, from about 1 day to about 5 days, from about 1 day to about 6 days, from about 1 day to about 7 days, from about 1 day to about 8 days, from about 1 day to about 9 days, from about 1 day to about 10 days, from about 1 day to about 12 days, from about 1 day to about 14 days, from about 1 day to about 3 weeks, or from about 1 day to about 4 weeks. The phase in which the DHODH inhibitor or other agent is withheld from the subject may be about 24 hours, about 36 hours, about 48 hours, about 60 hours, about 72 hours, about 84 hours, about 96 hours, about 5 days, about 6 days, about 7 days, about 8 days, about 9 days, about 10 days, about 12 days, about 2 weeks, about 3 weeks, about 4 weeks, about 5 weeks, about 6 weeks, about 8 weeks, about 2 months, about 3 months, about 4 months, about 5 months, about 6 months, from about 1 day to about 2 days, from about 1 day to about 3 days, from about 1 day to about 4 days, from about 1 day to about 5 days, from about 1 day to about 6 days, from about 1 day to about 1 week, from about 1 day to about 2 weeks, from about 1 day to about 3 weeks, from about 1 day to about 4 weeks, from about 1 day to about 6 weeks, from about 1 day to about 8 weeks, from about 1 day to about 2 months, from about 1 day to about 3 months, from about 1 day to about 4 months, from about 1 day to about 5 months, or from about 1 day to about 6 months.

The stages of a multistage treatment may be temporally discrete, or they may overlap in time. They may be separated by a gap during which neither the DHODH inhibitor nor the second therapeutic agent is provided to the subject. The stages and the gap may be of any suitable duration.

For example, each stage and the gap between stages may independently have a duration of about 24 hours, about 36 hours, about 48 hours, about 60 hours, about 72 hours, about 84 hours, about 96 hours, about 5 days, about 6 days, about 7 days, about 8 days, about 9 days, about 10 days, about 12 days, about 2 weeks, about 3 weeks, about 4 weeks, about 5 weeks, about 6 weeks, about 8 weeks, about 2 months, about 3 months, about 4 months, about 5 months, about 6 months, about 9 months, about 12 months, from about 1 day to about 2 days, from about 1 day to about 3 days, from about 1 day to about 4 days, from about 1 day to about 5 days, from about 1 day to about 6 days, from about 1 day to about 1 week, from about 1 day to about 2 weeks, from about 1 day to about 3 weeks, from about 1 day to about 4 weeks, from about 1 day to about 6 weeks, from about 1 day to about 8 weeks, from about 1 day to about 2 months, from about 1 day to about 3 months, from about 1 day to about 4 months, from about 1 day to about 5 months, or from about 1 day to about 6 months.

The different stages of the multistage treatment may be sequential. The stages and any gaps between them may make up a cycle, and the method may include multiple cycles. For example, the method may include two, three, four, five, six, or more cycles.

In a multistage treatment, the duration of any stage of a multistage treatment or the duration of any gap between stages may be determined by a response from the subject.

For example, the duration of a stage or a gap between stages may be determined by the subject's development of resistance to the DHODH inhibitor or the other agent. Resistance may be assessed by a change in a level of a biomarker in the subject. For example, the biomarker for resistance to the DHODH inhibitor may be N-carbamoylaspartate, dihydroorotate, orotate, orotidine 5′-monophosphate (OMP), or uridine monophoshpate (UMP). The biomarker for resistance to an IMPDH inhibitor may be guanosine triphosphate.

The duration of a stage or a gap between stages may be determined by the a change in a clinical outcome of the subject. A change in clinical outcome may include a change from one to another of the following disease states: active disease, minimal residual disease, remission, and complete molecular remission. For example and without limitation, disease states for acute myeloid leukemia (AML) may be as follows. Active disease may indicate that AML is still present during treatment or after treatment (refractory), that AML has come back after treatment (relapsed), or that a patient has more than 5% blast cells in the marrow. Minimal residual disease may indicate that AML cells are not detected in bone marrow using standard tests but are detected using more sensitive tests, such as flow cytometry or polymerase chain reaction (PCR). Remission may indicate that the patient has less than 5% blast cells in the marrow, that blood cell counts are within normal limits, or that the patient has no signs or symptoms of the disease. Complete molecular remission may indicate that AML cells are not detected in the marrow using sensitive tests, such as flow cytometry or polymerase chain reaction (PCR).

The duration of a stage or a gap between stages may be determined by another response of the subject. For example, the response may be change, such as an increase or decrease, in peripheral blood cell count, bone marrow blast count, or tumor burden.

The choice of second therapeutic agent may be determined by a response of the subject, such as any of the responses described above.

The dosing regimens, including the dosage and the dosing schedule, for the stages of a multistage treatment may be the same, or they may be different. For example, the first and second stages may differ in the dosage of the agent, the dosing schedule of the agent, both, or neither. For example and without limitation, the DHODH may be provided three times per day, two times per day, once per day, once every two days, once every three days, once every four days, once every five days, once every seven days, once every ten days, once every fourteen days, three times per week, two times per week, three times per month, or two times per month. For example and without limitation, the second agent may independently be provided three times per day, two times per day, once per day, once every two days, once every three days, once every four days, once every five days, once every seven days, once every ten days, once every fourteen days, three times per week, two times per week, three times per month, or two times per month. The multistage treatment may include any combination of the aforementioned dosing schedules.

### Measuring the Level of a Metabolite in a Sample

Methods of the invention may include analysis of a measured level of a metabolite, such as DHO in a sample from a subject. The methods may include measurement of the level of the metabolite.

In some embodiments, the metabolite is measured by mass spectrometry, optionally in combination with liquid chromatography. Molecules may be ionized for mass spectrometry by any method known in the art, such as ambient ionization, chemical ionization (CI), desorption electrospray ionization (DESI), electron impact (EI), electrospray ionization (ESI), fast-atom bombardment (FAB), field ionization, laser ionization (LIMS), matrix-assisted laser desorption ionization (MALDI), paper spray ionization, plasma and glow discharge, plasma-desorption ionization (PD), resonance ionization (RIMS), secondary ionization (SIMS), spark source, or thermal ionization (TIMS). Methods of mass spectrometry are known in the art and described in, for example, U.S. Pat. Nos. 8,895,918; 9,546,979; 9,761,426; Hoffman and Stroobant, Mass Spectrometry: Principles and Applications (2nd ed.). John Wiley and Sons (2001), ISBN 0-471-48566-7; Dass, Principles and practice of biological mass spectrometry, New York: John Wiley (2001) ISBN 0-471-33053-1; and Lee, ed., Mass Spectrometry Handbook, John Wiley and Sons, (2012) ISBN: 978-0-470-53673-5, the contents of each of which are incorporated herein by reference.

In certain embodiments, a sample can be directly ionized without the need for use of a separation system. In other embodiments, mass spectrometry is performed in conjunction with a method for resolving and identifying ionic species. Suitable methods include chromatography, capillary electrophoresis-mass spectrometry, and ion mobility. Chromatographic methods include gas chromatography, liquid chromatography (LC), high-pressure liquid chromatography (HPLC), and reversed-phase liquid chromatography (RPLC). In a preferred embodiment, liquid chromatography-mass spectrometry (LC-MS) is used. Methods of coupling chromatography and mass spectrometry are known in the art and described in, for example, Holcapek and Brydwell, eds. Handbook of Advanced Chromatography/Mass Spectrometry Techniques, Academic Press and AOCS Press (2017), ISBN 9780128117323; Pitt, Principles and Applications of Liquid Chromatography-Mass Spectrometry in Clinical Biochemistry, The Clinical Biochemist Reviews. 30(1): 19-34 (2017) ISSN 0159-8090; Niessen, Liquid Chromatography-Mass Spectrometry, Third Edition. Boca Raton: CRC Taylor & Francis. pp. 50-90. (2006) ISBN 9780824740825; Ohnesorge et al., Quantitation in capillary electrophoresis-mass spectrometry, Electrophoresis. 26 (21): 3973-87 (2005) doi:10.1002/elps.200500398; Kolch et al., Capillary electrophoresis-mass spectrometry as a powerful tool in clinical diagnosis and biomarker discovery, Mass Spectrom Rev. 24 (6): 959-77. (2005) doi:10.1002/mas.20051; Kanu et al., Ion mobility-mass spectrometry, Journal of Mass Spectrometry, 43 (1): 1-22 (2008) doi:10.1002/jms.1383, the contents of which are incorporated herein by reference.

A sample may be obtained from any organ or tissue in the individual to be tested, provided that the sample is obtained in a liquid form or can be pre-treated to take a liquid form. For example and without limitation, the sample may be a blood sample, a urine sample, a serum sample, a semen sample, a sputum sample, a lymphatic fluid sample, a cerebrospinal fluid sample, a plasma sample, a pus sample, an amniotic fluid sample, a bodily fluid sample, a stool sample, a biopsy sample, a needle aspiration biopsy sample, a swab sample, a mouthwash sample, a cancer sample, a tumor sample, a tissue sample, a cell sample, a synovial fluid sample, a phlegm sample, a saliva sample, a sweat sample, or a combination of such samples. The sample may also be a solid or semi-solid sample, such as a tissue sample, feces sample, or stool sample, that has been treated to take a liquid form by, for example, homogenization, sonication, pipette trituration, cell lysis etc. For the methods described herein, it is preferred that a sample is from plasma, serum, whole blood, or sputum.

The sample may be kept in a temperature-controlled environment to preserve the stability of the metabolite. For example, DHO is more stable at lower temperatures, and the increased stability facilitates analysis of this metabolite from samples. Thus, samples may be stored at, or 4° C., −20° C., or −80° C.

In some embodiments, a sample is treated to remove cells or other biological particulates. Methods for removing cells from a blood or other sample are well known in the art and may include e.g., centrifugation, sedimentation, ultrafiltration, immune selection, etc.

The subject may be an animal (such as a mammal, such as a human). The subject may be a pediatric, a newborn, a neonate, an infant, a child, an adolescent, a pre-teen, a teenager, an adult, or an elderly patient. The subject may be in critical care, intensive care, neonatal intensive care, pediatric intensive care, coronary care, cardiothoracic care, surgical intensive care, medical intensive care, long-term intensive care, an operating room, an ambulance, a field hospital, or an out-of-hospital field setting.

The sample may be obtained from an individual before or after administration to the subject of a DHODH inhibitor. For example, the sample may be obtained 1 hour, 2 hours, 4 hours, 6 hours, 8 hours, 12 hours, 24 hours, 36 hours, 2 days, 3 days, 4 days, 5 days, 6 days, 7 days or more before administration of a DHODH inhibitor, or it may be obtained 1 hour, 2 hours, 4 hours, 6 hours, 8 hours, 12 hours, 24 hours, 36 hours, 2 days, 3 days, 4 days, 5 days, 6 days, 7 days or more after administration of a DHODH inhibitor.

### Providing DHODH Inhibitors and Other Therapeutic Agents

Methods of the invention may include providing a DHODH inhibitor or other therapeutic agent to a subject according to a dosing regimen or dosage determined as described above. Providing the DHODH inhibitor or other therapeutic agent to the subject may include administering it to the subject. A dose may be administered as a single unit or in multiple units. The DHODH inhibitor or other therapeutic agent may be administered by any suitable means. For example and without limitation, the DHODH inhibitor or other therapeutic agent may be administered orally, intravenously, enterally, parenterally, dermally, buccally, topically, transdermally, by injection, intravenously, subcutaneously, nasally, pulmonarily, or with or on an implantable medical device (e.g., stent or drug-eluting stent or balloon equivalents).

In some embodiments, the methods include assessing the level of a metabolite, such as DHO, in a sample from a subject, and determining whether that level is within a threshold range (e.g., above a minimal threshold and/or below a potential toxicity threshold) that warrants dosing, and/or that warrants dosing at a particular level or in a particular amount.

The methods may include administering at least one dose of the DHODH inhibitor to a subject whose plasma DHO level has been determined and is below a pre-determined threshold (e.g., a pre-determined potential toxicity threshold and/or a pre-determined potential efficacy threshold). In some embodiments, the predetermined threshold reflects percent inhibition of DHODH in the subject relative to a baseline determined for the subject. In some embodiments, the baseline is determined by an assay.

For example, in some embodiments, in order to maintain inhibition of DHODH at an effective threshold, multiple doses of the DHODH inhibitor may be administered. In some embodiments, dosing of the DHODH inhibitor can occur at different times and in different amounts. The present disclosure encompasses those methods that can maintain inhibition of DHODH at a consistent level at or above the efficacy threshold throughout the course of treatment. In some embodiments, the amount of inhibition of DHODH is measured by the amount of DHO in the plasma of a subject.

In some embodiments, more than one dose of the DHODH inhibitor is administered to the subject. In some embodiments, the method further comprises a step of re-determining the subject's plasma DHO level after administration of the at least one dose. In some embodiments, the subject's plasma DHO level is re-determined after each dose. In some embodiments, the method further comprises administering at least one further dose of the DHODH inhibitor after the subject's plasma DHO level has been determined again (e.g., after administering a first or previous dose), and is below the pre-determined threshold. If the subject's plasma DHO level is determined to be above a pre-determined threshold, dosing can be discontinued. In some embodiments, therefore, no further dose of the DHODH inhibitor is administered until the subject's plasma DHO level has been determined to again be below a pre-determined threshold.

The methods may include administering a DHODH inhibitor to a subject at a dosage level at or near a cell-lethal level. Such dosage can be supplemented with a later dose at a reduced level, or by discontinuing of dosing. For example, in some embodiments, the present disclosure provides a method of administering a dihydroorotate dehydrogenase inhibitor to a subject in need thereof, the method comprising: administering a plurality of doses of a DHODH inhibitor, according to a regimen characterized by at least first and second phases, wherein the first phase involves administration of at least one bolus dose of a DHODH inhibitor at a cell-lethal level; and the second phase involves either: administration of at least one dose that is lower than the bolus dose; or absence of administration of a DHODH inhibitor.

In some embodiments, a DHODH inhibitor is not administered during a second phase. In some embodiments, a second phase involves administration of uridine rescue therapy. In some embodiments, a bolus dose is or comprises a cell lethal dose. In some embodiments, a cell lethal dose is an amount of a DHODH inhibitor that is sufficient to cause apoptosis in normal (e.g., non-cancerous) cells in addition to target cells (e.g., cancer cells).

In some embodiments, the first phase and the second phase each comprise administering a DHODH inhibitor. In some embodiments, the first phase and the second phase are at different times. In some embodiments, the first phase and the second phase are on different days. In some embodiments, the first phase lasts for a period of time that is less than four days. In some embodiments, the first phase comprises administering a DHODH inhibitor, followed by a period of time in which no DHODH inhibitor is administered. In some embodiments, the period of time in which no DHODH inhibitor is administered is 3 to 7 days after the dose during the first phase. In some embodiments, the first phase comprises administering more than one dose.

In some embodiments, a DHODH inhibitor is administered during a second phase. In some embodiments, a DHODH inhibitor is administered sub-cell-lethal levels during the second phase. In some embodiments, the first phase is repeated after the second phase. In some embodiments, both the first and second phases are repeated.

In some embodiments, the present disclosure provides a method of administering a DHODH inhibitor to a subject in need thereof, according to a multi-phase protocol comprising: a first phase in which at least one dose of the DHODH inhibitor is administered to the subject; and a second phase in which at least one dose of the DHODH inhibitor is administered to the subject, wherein one or more doses administered in the second phase differs in amount and/or timing relative to other doses in its phase as compared with the dose(s) administered in the first phase.

In some embodiments, a DHO level is determined in a sample from the subject between the first and second phases. In some embodiments, the sample is a plasma sample. In some embodiments, the timing or amount of at least one dose administered after the DHO level is determined or differs from that of at least one dose administered before the DHO level was determined.

In some embodiments, the amount of DHODH inhibitor that is administered to the patient is adjusted in view of the DHO level in the subject's plasma. For example, in some embodiments, a first dose is administered in the first phase. In some embodiments, DHO level is determined at a period of time after administration of the first dose.

In some embodiments, if the DHO level is below a pre-determined level, the amount of DHODH inhibitor administered in a second or subsequent dose is increased and/or the interval between doses is reduced. For example, in some such embodiments, the amount of DHODH inhibitor administered may be increased, for example, by 100 mg/m². In some embodiments, the amount of DHODH inhibitor administered in a second or subsequent dose is increased by 150 mg/m². In some embodiments, the amount of DHODH inhibitor administered in a second or subsequent dose is increased by 200 mg/m². In some embodiments, the amount of DHODH inhibitor administered may be increased by an adjustment amount determined based on change in DHO levels observed between prior doses of different amounts administered to the subject.

In some embodiments, if the DHO level is above a pre-determined level, the amount of DHODH inhibitor administered in a second or subsequent dose is the same as the amount administered in the first or previous dose and/or the interval between doses is the same.

In some embodiments, if the DHO level is above a pre-determined level, the amount of DHODH inhibitor in a second or subsequent dose is decreased and/or the interval between doses is increased. For example, in some such embodiments, the amount of DHODH inhibitor administered may be decreased, for example, by 50 mg/m². In some embodiments, if the DHO level is above a pre-determined level, the amount of DHODH inhibitor in a second or subsequent dose is decreased by 75 mg/m². In some embodiments, if the DHO level is above a pre-determined level, the amount of DHODH inhibitor in a second or subsequent dose is decreased by 100 mg/m². In some embodiments, the amount of DHODH inhibitor administered may be decreased by an adjustment amount determined based on change in DHO levels observed between prior doses of different amounts administered to the subject.

In some embodiments, the present disclosure provides a method of administering a later dose of a DHODH inhibitor to a patient who has previously received an earlier dose of the DHODH inhibitor, wherein the patient has had a level of DHO assessed subsequent to administration of the earlier dose, and wherein the later dose is different than the earlier dose. The later dose may be different from the earlier dose in amount of DHODH inhibitor included in the dose, time interval relative to an immediately prior or immediately subsequent dose, or combinations thereof. The amount of DHODH inhibitor in the later dose may be less than that in the earlier dose.

The method may include administering multiple dose of the DHODH inhibitor, separated from one another by a time period that is longer than 2 days and shorter than 8 days for example, the time period may be about 3 days.

In some embodiments, the DHO level is determined in a sample from the subject before each dose is administered, and dosing is delayed or skipped if the determined DHO level is above a pre-determined threshold. For example, the DHO level may be determined about 12 hours, about 24 hours, about 36 hours, about 48 hours, about 60 hours, about 72 hours, about 84 hours, or about 96 hours after administration of a DHODH inhibitor

The method may include administering the DHODH inhibitor according to a regimen approved in a trial in which a level of DHO was measured in a patients between doses of the DHODH inhibitor. The regimen may include multiple doses whose amount and timing were determined in the trial to maintain the DHO level within a range determined to indicate a degree of DHODH inhibition below a toxic threshold and above a minimum threshold. The regimen may include determining the DHO level in the subject after administration of one or more doses of the DHODH inhibitor.

In some embodiments, the regimen includes a dosing cycle in which an established pattern of doses is administered over a first period of time. In some embodiments, the regimen comprises a plurality of the dosing cycles. In some embodiments, the regimen includes a rest period during which the DHODH inhibitor is not administered between the cycles.

Disorders that can be Treated with Combination Therapies

The methods of the invention are useful for determining the dosage of drugs that affect that alter the activity of DHODH to treat or prevent a disorder. The disorder may be any disease, disorder, or condition for which DHODH inhibition provides a therapeutic benefit.

For example and without limitation, one disorder that can be treated by methods of the invention is acute myeloid leukemia (AML). In AML, myeloblasts arrested in an early stage of differentiation proliferate in an uncontrolled manner and interfere with the development of other blood cells in the bone marrow. Inhibitors of dihydroorotate dehydrogenase (DHODH), an enzyme involved in pyrimidine synthesis, cause differentiation of myeloblasts and prevent their leukemia-initiating activity. The role of DHODH in AML is described in Sykes et al., Inhibition of Dihydroorotate Dehydrogenase Overcomes Differentiation Blockade in Acute Myeloid Leukemia, Cell 167, 171-186, Sep. 22, 2016; dx.doi.org/10.1016/j.cell.2016.08.057, the contents of which are incorporate herein by reference.

The use of DHODH inhibitors to treat AML requires a precise dosing regimen. Care must be taken to avoid excessive inhibition of DHODH. DHODH is an essential enzyme, and homozygous recessive mutations in DHODH cause Miller syndrome, a disorder characterized by multi-organ dysfunction. In a mouse model of AML, daily administration of high doses of the DHODH inhibitor brequinar lead to weight loss, anemia, and thrombocytopenia. At the same time, sustained exposure to brequinar is necessary to inhibit DHODH for sufficient periods to produce a therapeutic effect in the mouse AML model. Without wishing to be bound by theory, one hypothesis for the narrow therapeutic window of brequinar in treating AML in both the mouse model and in humans is that malignant cells display an increased sensitivity to DHODH inhibition. In particular, normal cells may be able to tolerate periods of nucleotide starvation that kill cancer cells due to the elevated metabolic needs of the latter.

The narrow therapeutic window of DHODH inhibition likely applies to other disorders as well. For example, brequinar was evaluated for treatment of solid tumor malignancies and found to be ineffective when administered over a 5-day period followed by a 3-week gap or once per week for three weeks followed by a 1-week gap. See Arteaga, C. L. et al. (1989) Phase I clinical and pharmacokinetic trial of Brequinar sodium (DuP 785; NSC 368390) Cancer Res. 49, 4648-4653; Burris, H. A., et al. (1998) Pharmacokinetic and phase I studies of brequinar (DUP 785; NSC 368390) in combination with cisplatin in patients with advanced malignancies, Invest. New Drugs 16, 19-27; Noe, D. A., et al. (1990) Phase I and pharmacokinetic study of brequinar sodium (NSC 368390), Cancer Res. 50, 4595-4599; Schwartsmann, G. et al. (1990) Phase I study of Brequinar sodium (NSC 368390) in patients with solid malignancies, Cancer Chemother. Pharmacol. 25, 345-351, the contents of each of which are incorporated herein by reference. However, brequinar may be effective for treatment of other cancers if the drug is administered in a manner that provides sustained DHODH inhibition.

It is understood that the aforementioned examples are provided for illustrative purposes only and that the methods of the invention can be used for treatment of any disorder or disease in which the measured level of DHO can be used to assess target engagement. The disorder may be one in which inhibiting DHODH is of therapeutic benefit. The disorder may be cancer. The cancer may include a solid tumor or hematological tumor. The cancer may be acute lymphoblastic leukemia (ALL), adult T cell leukemia/lymphoma (ATLL), bladder cancer, breast cancer, such as triple negative breast cancer (TNBC), glioma, head and neck cancer, leukemia, such as AML, lung cancer, such as small cell lung cancer and non-small cell lung cancer, lymphoma, multiple myeloma, neuroblastoma, osteosarcoma, ovarian cancer, prostate cancer, or renal cell cancer. The disorder may have a genetic mutation such as MYC amplification or PTEN loss that leads to increased dependence on the metabolic pathway, such as increased “addiction” to glutamine. The disorder may be an inflammatory or autoimmune disorder, such as arthritis, hepatitis, chronic obstructive pulmonary disease, multiple sclerosis, or tendonitis. The disorder may be a psychiatric disorder, such as anxiety, stress, obsessive-compulsive disorder, depression, panic disorder, psychosis, addiction, alcoholism, attention deficit hyperactivity, agoraphobia, schizophrenia, or social phobia. The disorder may be a neurological or pain disorder, such as epilepsy, stroke, insomnia, diskinesia, peripheral neuropathic pain, chronic nociceptive pain, phantom pain, deafferentation pain, inflammatory pain, joint pain, wound pain, post-surgical pain, or recurrent headache pain, appetite disorders, or motor activity disorders. The disorder may be a neurodegenerative disorder, such as Alzheimer's disease, Parkinson's disease, or Huntington's disease.

The disorder may include a class or subset of patients having a disease, disorder, or condition. For example, AML cases are classified based on cytological, genetic, and other criteria, and AML treatment strategies vary depending on classification. One AML classification system is provided by the World Health Organization (WHO). The WHO classification system includes subtypes of AML provided in Table 1 and is described in Falini B, et al. (October 2010) “New classification of acute myeloid leukemia and precursor-related neoplasms: changes and unsolved issues” Discov Med. 10 (53): 281-92, PMID 21034669, the contents of which are incorporated herein by reference.

TABLE 1

| Name | Description |
|---|---|
| Acute myeloid | Includes: |
| leukemia with | AML with translocations between chromosome 8 and |
| recurrent | 21-[t(8;21)(q22;q22);] RUNX1/RUNX1T1; (ICD-O |
| genetic | 9896/3); |
| abnormalities | AML with inversions in chromosome 16- |
| | [inv(16)(p13.1q22)] or internal translocations in it- |
| | [t(16;16)(p13.1;q22);] CBFB/MYH11; (ICD-O |
| | 9871/3); |
| | Acute promyelocytic leukemia with translocations |
| | between chromosome 15 and 17- |
| | [t(15;17)(q22;q12);] RARA/PML; (ICD-O 9866/3); |
| | AML with translocations between chromosome 9 and |
| | 11-[t(9;11)(p22;q23);] MLLT3/MLL; |
| | AML with translocations between chromosome 6 and |
| | 9-[t(6;9)(p23;q34);] DEK/NUP214; |
| | AML with inversions in chromosome 3- |
| | [inv(3)(q21q26.2)] or internal translocations in it- |
| | [t(3;3)(q21;q26.2);] RPN1/EVI1; |
| | Megakaryoblastic AML with translocations between |
| | chromosome 1 and 22-[t(1;22)(p13;q13);] |
| | RBM15/MKL1; |
| | AML with mutated NPM1 |
| | AML with mutated CEBPA |
| AML with | Includes people who have had a prior documented |
| myelodysplasia- | myelodysplastic syndrome (MDS) or |
| related changes | myeloproliferative disease (MPD) that then has |
| | transformed into AML, or who have cytogenetic |
| | abnormalities characteristic for this type of AML (with |
| | previous history of MDS or MPD that has gone |
| | unnoticed in the past, but the cytogenetics is still |
| | suggestive of MDS/MPD history). This category of |
| | AML occurs most often in elderly people and often has |
| | a worse prognosis. Includes: |
| | AML with complex karyotype |
| | Unbalanced abnormalities |
| | AML with deletions of chromosome 7-[del(7q);] |
| | AML with deletions of chromosome 5-[del(5q);] |
| | AML with unbalanced chromosomal aberrations in |
| | chromosome 17-[i(17q)/t(17p);] |
| | AML with deletions of chromosome 13-[del(13q);] |
| | AML with deletions of chromosome 11-[del(11q);] |
| | AML with unbalanced chromosomal aberrations in |
| | chromosome 12-[del(12p)/t(12p);] |
| | AML with deletions of chromosome 9-[del(9q);] |
| | AML with aberrations in chromosome X- |
| | [idic(X)(q13);] |
| | Balanced abnormalities |
| | AML with translocations between chromosome 11 and |
| | 16-[t(11;16)(q23;q13.3);], unrelated to previous |
| | chemotherapy or ionizing radiation |
| | AML with translocations between chromosome 3 and |
| | 21-[t(3;21)(q26.2;q22.1);], unrelated to previous |
| | chemotherapy or ionizing radiation |
| | AML with translocations between chromosome 1 and |
| | 3-[t(1;3)(p36.3;q21.1);] |
| | AML with translocations between chromosome 2 and |
| | 11-[t(2;11)(p21;q23);], unrelated to previous |
| | chemotherapy or ionizing radiation |
| | AML with translocations between chromosome 5 and |
| | 12-[t(5;12)(q33;p12);] |
| | AML with translocations between chromosome 5 and |
| | 7-[t(5,7)(q33;q11.2);] |
| | AML with translocations between chromosome 5 and |
| | 17-[t(5;17)(q33;p13);] |
| | AML with translocations between chromosome 5 and |
| | 10-[t(5;10)(q33;q21);] |
| | AML with translocations between chromosome 3 and |
| | 5-[t(3;5)(q25;q34);] |
| Therapy-related | Includes people who have had prior chemotherapy |
| myeloid | and/or radiation and subsequently develop AML or |
| neoplasms | MDS. These leukemias may be characterized by |
| | specific chromosomal abnormalities, and often carry a |
| | worse prognosis. |
| Myeloid | Includes myeloid sarcoma. |
| sarcoma | |
| Myeloid | Includes so-called “transient abnormal myelopoiesis” |
| proliferations | and “Myeloid leukemia associated with Down |
| related to Down | syndrome” |
| syndrome | |
| Blastic | Includes so-called “blastic plasmacytoid dendritic cell |
| plasmacytoid | neoplasm” |
| dendritic cell | |
| neoplasm | |
| AML not | Includes subtypes of AML that do not fall into the |
| otherwise | above categories |
| categorized | AML with minimal differentiation |
| | AML without maturation |
| | AML with maturation |
| | Acute myelomonocytic leukemia |
| | Acute monoblastic and monocytic leukemia |
| | Acute erythroid leukemia |
| | Acute megakaryoblastic leukemia |
| | Acute basophilic leukemia |
| | Acute panmyelosis with myelofibrosis |

An alternative classification scheme for AML is the French-American-British (FAB) classification system. The FAB classification system includes the subtypes of AML provided in Table 2 and is described in Bennett J M, et al. (August 1976). “Proposals for the classification of the acute leukaemias. French-American-British (FAB) co-operative group” Br. J. Haematol. 33 (4): 451-8, doi:10.1111/j.1365-2141.1976.tb03563.x. PMID 188440; and Bennett J M, et al. (June 1989) “Proposals for the classification of chronic (mature) B and T lymphoid leukaemias. French-American-British (FAB) Cooperative Group” J. Clin. Pathol. 42 (6): 567-84, doi:10.1136/jcp.42.6.567, PMC 1141984, PMID 2738163, the contents of each of which are incorporated herein by reference.

TABLE 2

| Type | Name | Cytogenetics |
|---|---|---|
| M0 | acute myeloblastic leukemia, minimally | |
| | differentiated | |
| M1 | acute myeloblastic leukemia, without | |
| | maturation | |
| M2 | acute myeloblastic leukemia, with | t(8;21)(q22;q22), |
| | granulocytic maturation | t(6;9) |
| M3 | promyelocytic, or acute promyelocytic | t(15;17) |
| | leukemia (APL) | |
| M4 | acute myelomonocytic leukemia | inv(16)(p13q22), |
| | | del(16q) |
| M4eo | myelomonocytic together with bone | inv(16), t(16;16) |
| | marrow eosinophilia | |
| M5 | acute monoblastic leukemia (M5a) or | del(11q), t(9;11), |
| | acute monocytic leukemia (M5b) | t(11;19) |
| M6 | acute erythroid leukemias, including | |
| | erythroleukemia (M6a) and very rare pure | |
| | erythroid leukemia (M6b) | |
| M7 | acute megakaryoblastic leukemia | t(1;22) |

The disorder may include a sub-population of patients. For example, the patients may be pediatric, newborn, neonates, infants, children, adolescent, pre-teens, teenagers, adults, or elderly. The patients may be in critical care, intensive care, neonatal intensive care, pediatric intensive care, coronary care, cardiothoracic care, surgical intensive care, medical intensive care, long-term intensive care, an operating room, an ambulance, a field hospital, or an out-of-hospital field setting.

### EXAMPLES

### Example 1

Combination therapies of the invention were analyzed for their effectiveness for treating cancer in mice. Charles River (CR) 8 to 12 week old female NCI Ath/nu mice were injected subcutaneously in the flank with 1×10⁷ MV411 tumor cells in 50% Matrigel. The injection volume was 0.1 mL/mouse. When the tumors reached 100-150 mm³ groups (n=10) were pair matched and treatment began. Brequinar was dosed orally every three days at 50 mg/kg. Ribavirin was dosed orally daily at either 50 mg/kg or 100 mg/kg. Combinations of brequinar and ribavirin were dosed on those same schedules. Tumor volume and animal weight was measured twice weekly.

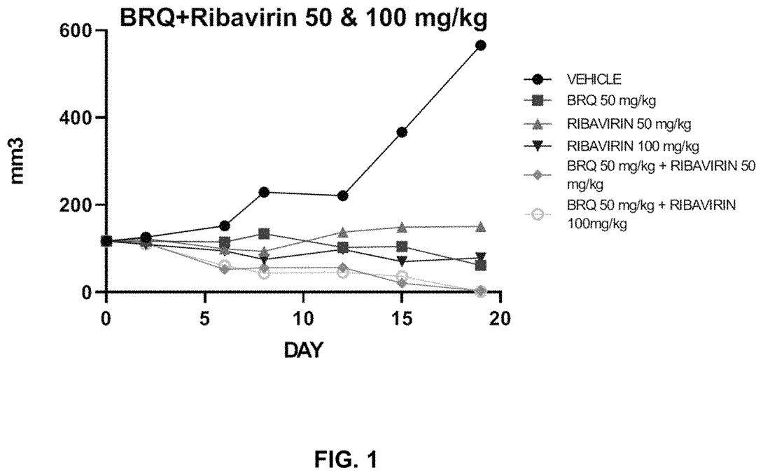

FIG. 1 is a graph showing mean tumor volume in mice treated with vehicle, brequinar, ribavirin, or combinations of brequinar and ribavirin. Solid circles represent values from mice treated with vehicle alone; squares represent values from mice treated with brequinar alone; upward-pointing triangles represent values from mice treated with 50 mg/kg ribavirin alone; downward-pointing triangles represent values from mice treated with 100 mg/kg ribavirin alone; diamonds represent values from mice treated with a combination of brequinar and 50 mg/kg ribavirin; and open circles represent values from mice treated with a combination of brequinar and 100 mg/kg ribavirin.

The data show that treatment with a combination of brequinar and ribavirin causes a rapid inhibition in tumor growth and a decrease in size of the original tumor. Thus, the results suggest that a combination that includes a DHODH inhibitor and a second agent that targets a different metabolic pathway is therapeutically superior to either agent alone.

### Example 2

Combination therapies of the invention were analyzed for their effectiveness for treating cancer in mice. Charles River (CR) 8 to 12 week old female NCI Ath/nu mice were injected subcutaneously in the flank with 1×10⁷ MV411 tumor cells in 50% Matrigel. The injection volume was 0.1 mL/mouse. When the tumors reached 100-150 mm³ groups (n=10) were pair matched and treatment began. Brequinar was dosed orally every three days at 50 mg/kg. Venetoclax was dosed orally daily at either 50 mg/kg or 100 mg/kg. Combinations of brequinar and venetoclax were dosed on those same schedules. Tumor volume and animal weight was measured twice weekly.

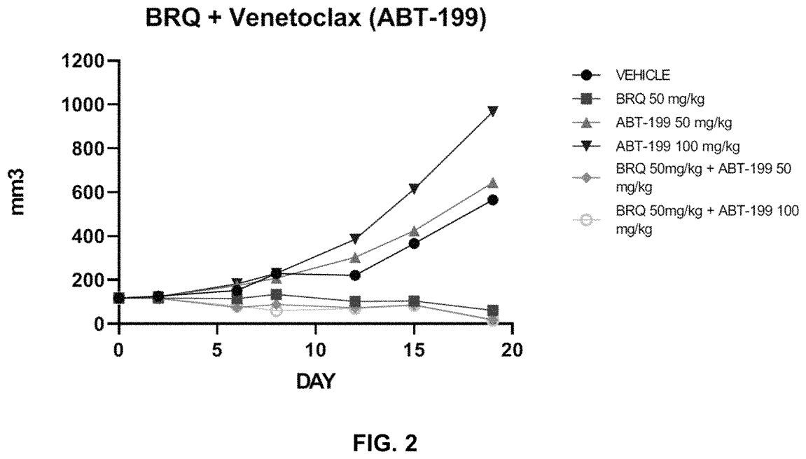

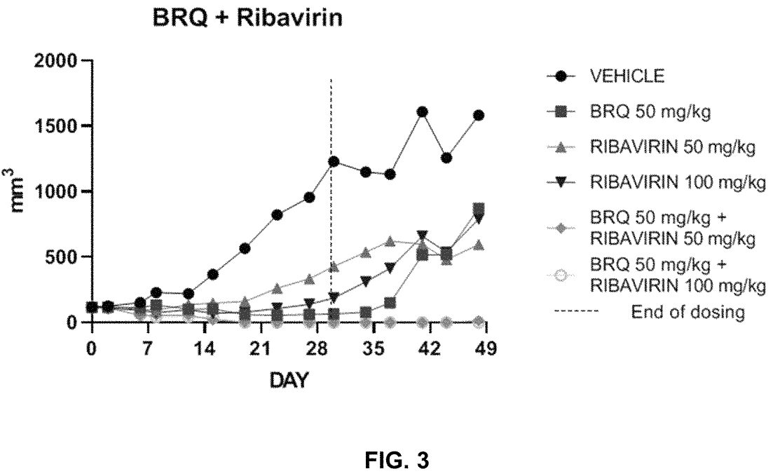

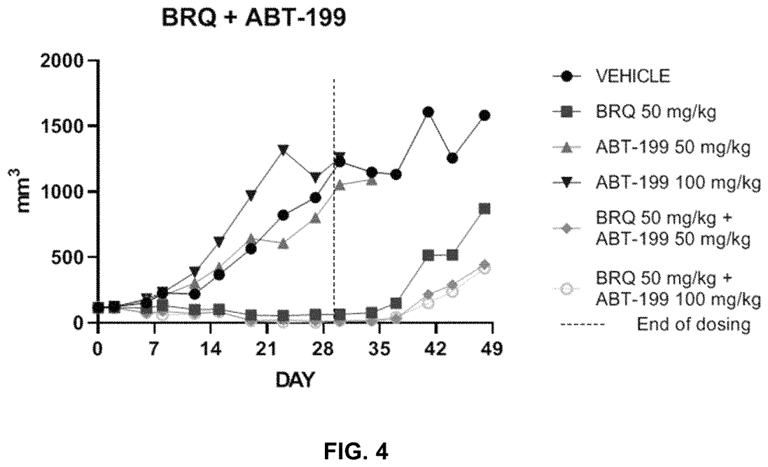

FIG. 2 is a graph showing the mean tumor volume in mice treated with vehicle, brequinar, venetoclax, or combinations of brequinar and venetoclax. Solid circles represent values from mice treated with vehicle alone; squares represent values from mice treated with brequinar alone; upward-pointing triangles represent values from mice treated with 50 mg/kg venetoclax (ABT-199) alone; downward-pointing triangles represent values from mice treated with 100 mg/kg venetoclax (ABT-199) alone; diamonds represent values from mice treated with a combination of brequinar and 50 mg/kg venetoclax (ABT-199); and open circles represent values from mice treated with a combination of brequinar and 100 mg/kg/day venetoclax (ABT-199).

The data show that treatment with a combination of brequinar and venetoclax causes a rapid inhibition in tumor growth and a decrease in size of the original tumor. Thus, the results suggest that a combination that includes a DHODH inhibitor and a second agent that targets a different metabolic pathway is therapeutically superior to either agent alone.

### Example 3

Combination therapies of the invention were analyzed for their effectiveness for treating cancer in mice as described in Example 1.

FIG. 3 is a graph showing mean tumor volume in mice treated with vehicle, brequinar, ribavirin, or combinations of brequinar and ribavirin. Solid circles represent values from mice treated with vehicle alone; squares represent values from mice treated with brequinar alone; upward-pointing triangles represent values from mice treated with 50 mg/kg ribavirin alone; downward-pointing triangles represent values from mice treated with 100 mg/kg ribavirin alone; diamonds represent values from mice treated with a combination of brequinar and 50 mg/kg ribavirin; and open circles represent values from mice treated with a combination of brequinar and 100 mg/kg ribavirin.

The data show that treatment with a combination of brequinar and ribavirin causes a rapid inhibition in tumor growth and a decrease in size of the original tumor. Thus, the results suggest that a combination that includes a DHODH inhibitor and a second agent that targets a different metabolic pathway is therapeutically superior to either agent alone.

### Example 4

Combination therapies of the invention were analyzed for their effectiveness for treating cancer in mice as described in Example 2.

FIG. 4 is a graph showing the mean tumor volume in mice treated with vehicle, brequinar, venetoclax, or combinations of brequinar and venetoclax. Solid circles represent values from mice treated with vehicle alone; squares represent values from mice treated with brequinar alone; upward-pointing triangles represent values from mice treated with 50 mg/kg venetoclax (ABT-199) alone; downward-pointing triangles represent values from mice treated with 100 mg/kg venetoclax (ABT-199) alone; diamonds represent values from mice treated with a combination of brequinar and 50 mg/kg venetoclax (ABT-199); and open circles represent values from mice treated with a combination of brequinar and 100 mg/kg/day venetoclax (ABT-199).

The data show that treatment with a combination of brequinar and venetoclax causes a rapid inhibition in tumor growth and a decrease in size of the original tumor. Thus, the results suggest that a combination that includes a DHODH inhibitor and a second agent that targets a different metabolic pathway is therapeutically superior to either agent alone.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

1. A combination therapy for treatment of cancer in a subject, the combination therapy comprising:
a dihydroorotate dehydrogenase (DHODH) inhibitor; and
an agent selected from the group consisting of an inosine monophosphate dehydrogenase (IMPDH) inhibitor, a hypoxanthine-guanine phosphoribosyltransferase (HGPRT) inhibitor, a dihydrofolate reductase (DHFR) inhibitor, a Bcl-2 inhibitor, an agent that targets a cell-surface marker, a cytokine receptor inhibitor, a DNA methylation inhibitor, a DNA polymerase inhibitor, a DNA alkylating agent, an analog of a nucleoside or nucleobase, a topoisomerase inhibitor, an inhibitor of a Hedgehog signaling pathway, a tyrosine kinase inhibitor, a phosphoinositide 3-kinase (PI3-kinase) inhibitor, an inhibitor of eukaryotic initiation factor 4A (eIF4A), and an asparaginase.

2. The combination therapy of claim 1, wherein the DHODH inhibitor is selected from the group consisting of brequinar, leflunomide, and teriflunomide, wherein each of said DHODH inhibitors includes analogs, derivatives, prodrugs, micellar formulations, sustained release formulations, and salts thereof.

3. The combination therapy of claim 2, wherein the DHODH inhibitor is brequinar or an analog, derivative, prodrug, micellar formulation, sustained release formulation, or salt thereof.

4. The combination therapy of claim 1, wherein the agent is an IMPDH inhibitor selected from the group consisting of mizoribine, mycophenolic acid, ribavirin, selenazofurin, taribavirin, and tiazofurin, wherein each of said IMPDH inhibitors includes analogs, derivatives, prodrugs, micellar formulations, sustained release formulations, and salts thereof.

5. The combination therapy of claim 4, wherein the IMPDH inhibitor is tiazofurin or an analog, derivative, prodrug, micellar formulation, sustained release formulation, or salt thereof.

6. The combination therapy of claim 4, wherein the IMPDH inhibitor is ribavirin or an analog, derivative, prodrug, micellar formulation, sustained release formulation, or salt thereof.

7. The combination therapy of claim 1, wherein the agent is a Bcl-2 inhibitor.

8. The combination therapy of claim 7, wherein the Bcl-2 inhibitor is venetoclax or an analog, derivative, prodrug, micellar formulation, sustained release formulation, or salt thereof.

9. The combination therapy of claim 1, wherein the agent is a DNA methylation inhibitor.

10. The combination therapy of claim 9, wherein the DNA methylation inhibitor is azacitidine or an analog, derivative, prodrug, micellar formulation, sustained release formulation, or salt thereof.

11. The combination therapy of claim 1, wherein the agent is a DNA polymerase inhibitor.

12. The combination therapy of claim 11, wherein the DNA polymerase inhibitor is cytarabine or an analog, derivative, prodrug, micellar formulation, sustained release formulation, or salt thereof.

13. The combination therapy of claim 1, wherein:
the DHODH inhibitor is brequinar or an analog, derivative, prodrug, micellar formulation, sustained release formulation, or salt thereof; and
the agent is ribavirin or an analog, derivative, prodrug, micellar formulation, sustained release formulation, or salt thereof.

14. The combination therapy of claim 1, wherein:
the DHODH inhibitor is brequinar or an analog, derivative, prodrug, micellar formulation, sustained release formulation, or salt thereof; and
the agent is venetoclax or an analog, derivative, prodrug, micellar formulation, sustained release formulation, or salt thereof.

15. A method for treating cancer in a subject, the method comprising providing to a subject having cancer:
a dihydroorotate dehydrogenase (DHODH) inhibitor; and
an agent selected from the group consisting of an inosine monophosphate dehydrogenase (IMPDH) inhibitor, a hypoxanthine-guanine phosphoribosyltransferase (HGPRT) inhibitor, a dihydrofolate reductase (DHFR) inhibitor, a Bcl-2 inhibitor, an agent that targets a cell-surface marker, a cytokine receptor inhibitor, a DNA methylation inhibitor, a DNA polymerase inhibitor, a DNA alkylating agent, an analog of a nucleoside or nucleobase, a topoisomerase inhibitor, an inhibitor of a Hedgehog signaling pathway, a tyrosine kinase inhibitor, a phosphoinositide 3-kinase (PI3-kinase) inhibitor, an inhibitor of eukaryotic initiation factor 4A (eIF4A), and an asparaginase.

16. The method of claim 15, wherein the DHODH inhibitor and the agent are provided according to different dosing regimens.

17. The method of claim 15, wherein the DHODH inhibitor is provided orally or intravenously.

18. The method of claim 15, wherein the agent is provided orally, intravenously, or subcutaneously.

19. The method of claim 15, wherein the agent is provided to the subject during a first phase having a duration of from 1 day to 14 days and is withheld from the subject during a second phase having a duration of from 1 day to about 3 months.

20. The method of claim 15, wherein the DHODH inhibitor is selected from the group consisting of brequinar, leflunomide, and teriflunomide, wherein each of said DHODH inhibitors includes analogs, derivatives, prodrugs, micellar formulations, sustained release formulations, and salts thereof.

21-47. (canceled)
